(19) United States
(12) Patent Application Publication
(10) Pub. No.: US 2024/0132821 A1
(43) Pub. Date: Apr. 25, 2024

(54) Support Device And Cell Culturing System

(71) Applicants: Terumo Kabushiki Kaisha, Tokyo (JP); Terumo BCT, Inc., Lakewood, CO (US)

(72) Inventor: Masatsugu IGARASHI, Kanagawa (JP)

(21) Appl. No.: 18/402,047

(22) Filed: Jan. 2, 2024

Related U.S. Application Data
(63) Continuation of application No. PCT/JP2022/035073, filed on Sep. 21, 2022.

(30) Foreign Application Priority Data
Sep. 29, 2021 (JP) 2021-160269

(51) Int. Cl. C12M 3/00; C12M 1/26

(52) U.S. Cl. CPC C12M 23/48; C12M 33/00

(57) ABSTRACT

A cell culturing system is equipped with a cell culturing device and a support device. The cell culturing device comprises a culturing unit and a sampling unit. The support device includes a housing and a sampling support unit. The housing includes an accommodation chamber in which the culturing unit is accommodated. A sampling circuit unit is capable of being attached to and detached from the sampling support unit. The sampling support unit is installed on the outer surface of the housing.

### CROSS-REFERENCE TO RELATED APPLICATIONS

The present application is a continuation application of the International Patent Application No. PCT/JP2022/035073 filed Sep. 21, 2022, which designated the U.S., and claims the benefit of priority from Japanese Patent Application No. JP2021-160269 filed Sep. 30, 2021. The entire disclosure of the above-identified applications are incorporated herein by reference.

### FIELD

The present disclosure relates to a support device and a cell culturing system.

### BACKGROUND

A cell culturing system is often equipped with a cell culturing device and a support device, where the support device is configured to support the cell culturing device. The cell culturing device may include a bioreactor and a sampling unit, such as detailed, for example, in U.S. Pat. No. 9,442,047. The bioreactor may be configured for the culturing of cell. The sampling unit may be configured to collect a culture medium used for culturing the cells.

It is often necessary for the cell culturing system to be arranged inside a clean room. Accordingly, it is often desirable for support devices to be as compact as possible.

### SUMMARY

In at least one example embodiment, the present disclosure provides a support device that is configured to support a cell culturing device, where the cell culturing device includes a culturing unit and a sampling unit. The culturing unit may include a bioreactor equipped to culture cells. The sampling unit may be configured to collect from the culturing unit a culture medium used for culturing the cells. The sampling unit may include a sampling circuit unit and a sampling flow path configured to mutually connect the culturing unit and the sampling circuit unit. The support device may include a housing that includes an accommodation chamber in which the culturing unit is accommodated and a sampling support unit to which the sampling circuit unit is capable of being attached and detached. The sampling support unit may be installed on an outer surface of the housing.

In at least one example embodiment, the present disclosure is characterized by a cell culturing system including the aforementioned support device and the cell culturing device.

In at least one example embodiment, the sampling support unit may be installed on the outer surface of the housing. As a result, the support device may be made more compact as compared, for example, to a case in which a sampling support unit is arranged at a location separated away from a housing. Further, the sampling flow path in accordance with at least one example embodiment of the present disclosure may be short, so as to allow the amount of the culture medium collected in the sampling unit can be reduced as compared, for example, to a case in which a sampling support unit is arranged at a location separated away from a housing.

### BRIEF DESCRIPTION OF DRAWINGS

FIG. 1 is a schematic circuit diagram of an example cell culturing system in accordance with at least one example embodiment of the present disclosure;

FIG. 2 is a schematic circuit diagram of an example culture processing unit in accordance with at least one example embodiment of the present disclosure;

FIG. 3 is a perspective view of the cell culturing system introduced in FIG. 1, where a door of the cell culturing system is in a closed position in accordance with at least one example embodiment of the present disclosure;

FIG. 4 is a perspective view of the cell culturing system introduced in FIG. 1, where a door of the cell culturing system is in an opened position in accordance with at least one example embodiment of the present disclosure; and

FIG. 5 is a perspective view of another example cell culturing system in accordance with at least one example embodiment of the present disclosure.

### DETAILED DESCRIPTION

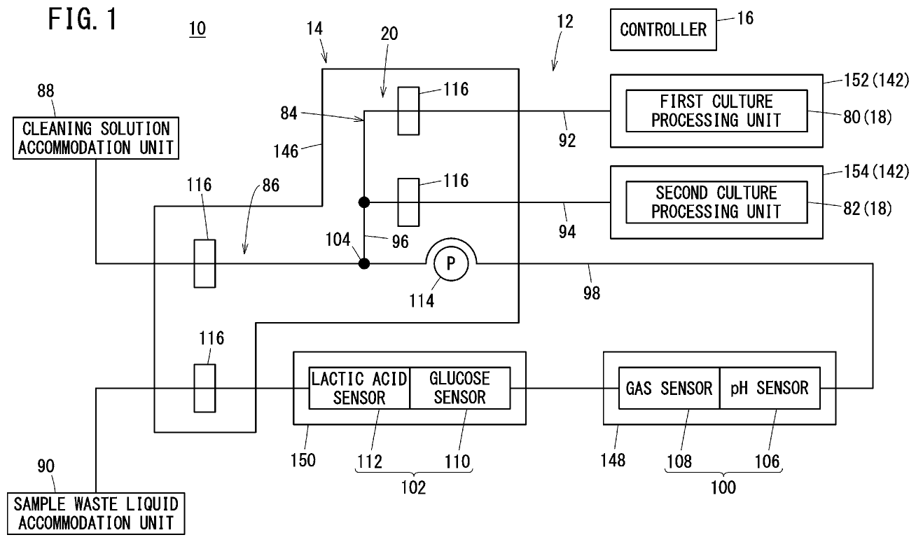

FIG. 1 illustrates an example cell culturing system 10. The cell culturing system 10 is configured to culture (propagates), within a culture medium, cells that have been separated from biological tissue. The cells used in the cell culturing system 10 may include adherent cells, planktonic cells, or a combination of adherent cells and planktonic cells. The cells used in the cell culturing system 10 may include, for example, ES cells, iPS cells, mesenchymal stem cells, or the like, or any combination thereof. The cells to be cultured using the cell culturing system 10 are not limited to the cell types described above.

The cell culturing system 10 includes a cell culturing device 12, a support device 14, and a controller 16. A liquid including, for example, a cell solution, a culture medium, a cleaning solution, a stripping solution, or any combination thereof may flow through the cell culturing device 12.

The cell solution may be a solution that contains cells. The culture medium may cause the cells in contact therewith to propagate. The culture medium may be selected depending on the cells to be cultured. The culture medium may include, for example, an MEM (Minimum Essential Medium), a Balanced Salt Solution (BSS), an FBS (fetal bovine serum), MEM-alpha with L-alanine-L-glutamine dipeptide (GlutaMAX), or the like, or any combination thereof. The cleaning solution may be selected to clean the interior of the cell culturing device 12. The cleaning solution may include, for example, water, a buffer solution, a physiological saline solution, or the like, or any combination thereof. The buffer solution may include, for example, PBS (Phosphate Buffered Salts), TBS (Tris-Buffered Saline), or the like, or any combination thereof. The stripping solution may be selected to strip the cells from later-described bioreactors 30 of the cell culturing device 12. The stripping solution may include, for example, trypsin, an EDTA solution, or any combination thereof. The culture medium, the cleaning solution, and the stripping solution are not limited to those described above.

The cell culturing device 12 may be discarded after being used one time (e.g., every time that a predetermined number of cells have been cultured). That is, the cell culturing device 12 may be a disposable product. In at least one example embodiment, the cell culturing device 12 may include two culture processing units 18 and one sampling unit 20.

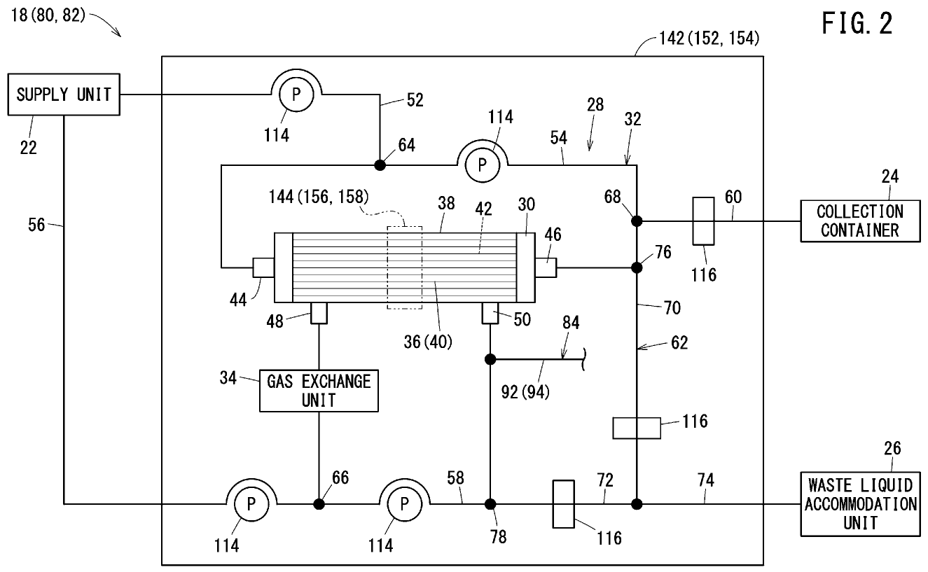

As illustrated in FIG. 2, each of the culture processing units 18 may include a supply unit 22, a collection container 24, a waste liquid accommodation unit 26, and a culturing unit 28.

The supply unit 22 may be configured to supply the cell solution, the culture medium, the cleaning solution, and the stripping solution to the culturing unit 28. The collection container 24 may be configured to collect the cells that are cultured in the culturing unit 28. The waste liquid accommodation unit 26 may be configured to accommodate the waste liquid that is generated in the culturing unit 28. In at least one example embodiment, the collection container 24 and/or the waste liquid accommodation unit 26 may include a medical bag obtained by molding a soft resin material into a bag-like shape. The soft resin material may include, for example, polyvinyl chloride, polyolefin, or the like, or any combination thereof. In other example embodiment, the collection container 24 and/or the waste liquid accommodation unit 26 may include tank or the like, such as, prepared, for example, using a hard resin.

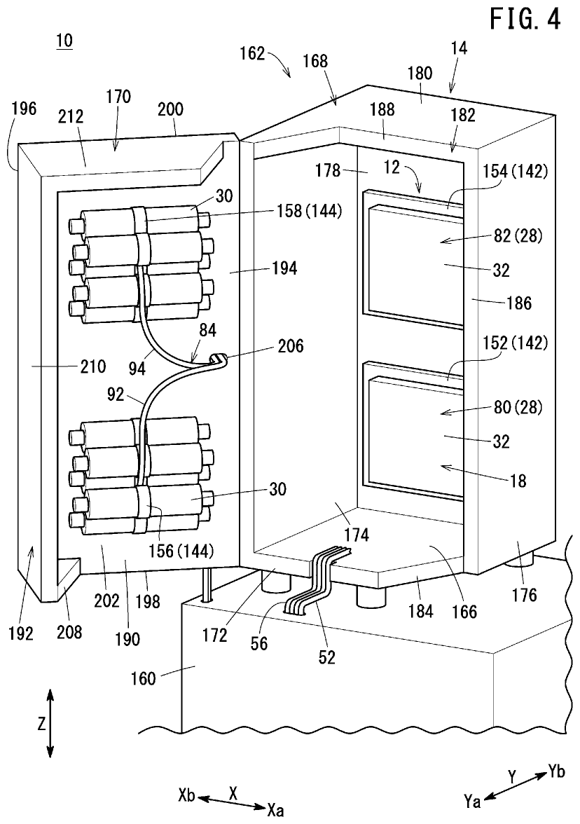

The culturing unit 28 may include a plurality of bioreactors 30 (see FIG. 4), a culturing circuit 32, and a gas exchange unit 34. It should be noted that, in FIG. 2, only one of the bioreactors 30 is illustrated for simplicity.

Each of the bioreactors 30 may include a plurality of hollow fiber membranes 36 and a cylindrical housing 38. The plurality of hollow fiber membranes 36 may be accommodated inside the housing 38. For example, a first end of each respective hollow fiber membrane 36 may be fixed to one end of the housing 38, and a second end of each respective hollow fiber membranes 36 may be fixed to another (or second) end of the housing 38.

The respective hollow fiber membranes 36 may be formed using a polymer material. The polymer material may include, for example, polypropylene, polyolefin resin, polysulfone, polyether sulfone, polyacrylonitrile, polytetrafluoroethylene, polystyrene, polymethylmethacrylate, cellulose acetate, cellulose triacetate, regenerated cellulose, or the like, or any combination thereof. However, the material constituting the respective hollow fiber membranes 36 is not limited to the aforementioned materials.

Each of the bioreactors 30 may include a first region 40 and a second region 42. The first region 40 may be defined by inner holes of the plurality of hollow fiber membranes 36. The second region 42 may be defined by a space between an inner peripheral surface of the housing 38 and outer peripheral surfaces of the plurality of hollow fiber membranes 36. Each of the hollow fiber membranes 36 may include a plurality of non-illustrated pores therein. The first region 40 and the second region 42 may communicate with each other through the plurality of pores of the respective hollow fiber membranes 36. The diameter of the pores may be of a size that allows small molecules (for example, water, ions, oxygen, lactic acid, etc.) to pass therethrough, while preventing the passage of macromolecules (for example, cells, etc.) therethrough. For example, in at least one example embodiment, the diameter of the respective pores may be greater than or equal to 0.005 micrometers and less than or equal to 10 micrometers.

A first inlet port 44, a first outlet port 46, a second inlet port 48, and a second outlet port 50 may be installed in the housing 38. The first inlet port 44 may be installed at one end (e.g., a first end) of the housing 38. The first inlet port 44 may be configured to communicate with the first region 40 via an inlet positioned at one end of the plurality of hollow fiber membranes 36. The first outlet port 46 may be installed at another end (e.g., a second end) of the housing 38. The first outlet port 46 may be configured to communicate with the first region 40 via an outlet positioned at the other end of the plurality of hollow fiber membranes 36.

The second inlet port 48 and the second outlet port 50 may be installed on an outer peripheral surface of the housing 38. The second inlet port 48 may be positioned between a center of the housing 38 and the first inlet port 44 in the longitudinal direction of the housing 38. The second outlet port 50 may be positioned between the center of the housing 38 and the first outlet port 46 in the longitudinal direction of the housing 38. Each of the second inlet port 48 and the second outlet port 50 may be configured to communicate with the second region 42.

The culturing circuit 32 may include flow paths which are extended in a linear shape. In at least one example embodiment, the culturing circuit 32 may include a plurality of tubes through which the liquids flow. The respective tubes may be formed of a soft resin material.

The culturing circuit 32 is not limited to the configuration described above. The culturing circuit 32 may include, for example, a sheet member including the flow paths therein through which the liquids flow. The sheet member may include two sheets made of a soft resin material which are stacked on each other in a thickness direction. Locations within the two sheets other than portions thereof that make up the flow paths may be joined (e.g., fusion bonded) mutually to each other. Within the two sheets, flow path wall parts that make up the flow paths may not be joined to each other. Within the sheet member, the flow path wall parts preferably bulge outward in a natural state in which liquid is not flowing through the flow paths. Extra portions on both sides of the sheet member in directions intersecting the flow paths may be cut off.

The culturing circuit 32 may include a first supply flow path 52, a first circulation flow path 54, a second supply flow path 56, a second circulation flow path 58, a collection flow path 60, and a waste liquid flow path 62. One end (e.g., a first end) of the first supply flow path 52 may be connected to the supply unit 22. The supply unit 22 may be configured to supply the cell solution, the culture medium, the cleaning solution, and the stripping solution one at a time at a predetermined timing to the first supply flow path 52. Another end (e.g., a second end) of the first supply flow path 52 may merge with the first circulation flow path 54.

A first merging section 64, which is a portion within the first circulation flow path 54 to which the first supply flow path 52 is connected, may be positioned at an intermediate portion in a direction in which the first circulation flow path 54 extends. Although not specifically illustrated, it should be appreciated that, in at least one example embodiment, the first circulation flow path 54 may include a plurality of one end parts. The plurality of one end parts of the first circulation flow path 54 may be connected respectively to each of the first inlet ports 44 of the plurality of bioreactors 30. The first circulation flow path 54 may include a plurality of other end parts. The plurality of other end parts of the first circulation flow path 54 may be connected respectively to each of the first outlet ports 46 of the plurality of bioreactors 30. The first circulation flow path 54 may communicate with the inner holes (e.g., the first region 40) of the plurality of hollow fiber membranes 36. In at least one example embodiment, the plurality of bioreactors 30 may be installed in parallel with the first circulation flow path 54.

One end (e.g., a first end) of the second supply flow path 56 may be connected to the supply unit 22. The supply unit 22 may be configured to supply the culture medium and the cleaning solution one at a time at a predetermined timing to the second supply flow path 56. Another end (e.g., a second end) of the second supply flow path 56 may merge with the second circulation flow path 58.

A second merging section 66, which is a portion within the second circulation flow path 58 to which the second supply flow path 56 is connected, may be positioned at an intermediate portion in a direction in which the second circulation flow path 58 extends. Although not specifically illustrated, it should be appreciated that, in at least one example embodiment, the second circulation flow path 58 may include a plurality of one end parts. The plurality of one end parts of the second circulation flow path 58 may be connected respectively to each of the second inlet ports 48 of the plurality of bioreactors 30. The second circulation flow path 58 may include a plurality of other end parts. The plurality of other end parts of the second circulation flow path 58 may be connected respectively to each of the second outlet ports 50 of the plurality of bioreactors 30. The second circulation flow path 58 may communicate with the space (e.g., the second region 42) between the plurality of hollow fiber membranes 36 and the housing 38.

The collection flow path 60 may extend from the first circulation flow path 54. A collection branching section 68, which is a portion within the first circulation flow path 54 to which the collection flow path 60 is connected, may be positioned in the first circulation flow path 54 between the first merging section 64 and the first outlet ports 46 of the plurality of bioreactors 30. An extending end of the collection flow path 60 may be connected to the collection container 24.

The waste liquid flow path 62 may be a flow path for discarding the liquids that flow through the first circulation flow path 54 and the second circulation flow path 58. The waste liquid flow path 62 may include a first waste liquid flow path 70, a second waste liquid flow path 72, and a third waste liquid flow path 74. The first waste liquid flow path 70 may extend from the first circulation flow path 54. Within the first circulation flow path 54, a first branching section 76, which is a portion to which the first waste liquid flow path 70 is connected, may be positioned between the collection branching section 68 and the first outlet ports 46 of the plurality of bioreactors 30 in the first circulation flow path 54.

The second waste liquid flow path 72 may extend from the second circulation flow path 58. Within the second circulation flow path 58, a second branching section 78, which is a portion to which the second waste liquid flow path 72 is connected, may be positioned between the second merging section 66 and the second outlet ports 50 of the plurality of bioreactors 30 in the second circulation flow path 58.

An extending end of the first waste liquid flow path 70 and an extending end of the second waste liquid flow path 72 may be connected to one end (e.g., a first end) of the third waste liquid flow path 74. Another end (e.g., a second end) of the third waste liquid flow path 74 may be connected to the waste liquid accommodation unit 26.

The gas exchange unit 34 may be installed between the second merging section 66 and the second inlet ports 48 of the plurality of bioreactors 30 in the second circulation flow path 58. The gas exchange unit 34 may allow a gas having predetermined components to pass through the liquid (the culture medium) that flows through the second circulation flow path 58. The gas used in the gas exchange unit 34 may include, for example, components therein that are similar to those of natural air. That is, the gas may include nitrogen, oxygen, carbon dioxide, or any combination thereof. In at least one example embodiment, the gas may include, for example, 75% nitrogen, 20% oxygen, and 5% carbon dioxide by volume.

In the following description, one of the two culture processing units 18 may be referred to as a first culture processing unit 80 and the other of the two culture processing units 18 may be referred to as a second culture processing unit 82.

As illustrated in FIG. 1, the sampling unit 20 may include a sampling flow path 84, a sampling circuit unit 86, a cleaning solution accommodation unit 88, and a sample waste liquid accommodation unit 90. The sampling flow path 84 may include a first introduction flow path 92, a second introduction flow path 94, and a third introduction flow path 96.

One end (e.g., a first end) of the first introduction flow path 92 may be connected to the first culture processing unit 80. In at least one example embodiment, within the first culture processing unit 80, the one end of the first introduction flow path 92 may be connected between the second branching section 78 and the second outlet ports 50 of the plurality of bioreactors 30 in the second circulation flow path 58 (see FIG. 2). A non-illustrated aseptic filter may be installed in the first introduction flow path 92. Another end (e.g., a second end) of the first introduction flow path 92 may be connected to one end of the third introduction flow path 96.

One end (e.g., a first end) of the second introduction flow path 94 may be connected to the second culture processing unit 82. In at least one example embodiment, within the second culture processing unit 82, the one end of the second introduction flow path 94 may be connected between the second branching section 78 and the second outlet ports 50 of the plurality of bioreactors 30 in the second circulation flow path 58 (see FIG. 2). A non-illustrated aseptic filter may be installed in the second introduction flow path 94. Another end (e.g., a second end) of the second introduction flow path 94 may be connected to one end (e.g., a first end) of the third introduction flow path 96.

The sampling circuit unit 86 may include a measurement flow path 98, a first sensor unit 100, and a second sensor unit 102. One end (e.g., a first end) of the measurement flow path 98 may be connected to the cleaning solution accommodation unit 88. Another end (e.g., a second end) of the measurement flow path 98 may be connected to the sample waste liquid accommodation unit 90. Another end (e.g., a second end) of the third introduction flow path 96 is connected to the measurement flow path 98.

The first sensor unit 100 may be installed in the measurement flow path 98. The first sensor unit 100 may be positioned in the measurement flow path 98 between the third merging section 104 where the other end of the third introduction flow path 96 merges and the sample waste liquid accommodation unit 90. The first sensor unit 100 may be an integrally molded product. The first sensor unit 100 may include a pH sensor 106 and a gas sensor 108.

The pH sensor 106 may be configured to measure a pH (hydrogen ion index) of the liquid flowing through the measurement flow path 98. The gas sensor 108 may be configured to measure a gas concentration of the liquid flowing through the measurement flow path 98. In at least one example embodiment, the gas sensor 108 may include an oxygen sensor and/or a carbon dioxide sensor. The oxygen sensor may be configured to measure an oxygen concentration of the liquid flowing through the measurement flow path 98. The carbon dioxide sensor may be configured to measure a carbon dioxide concentration of the liquid flowing through the measurement flow path 98.

The second sensor unit 102 may be installed in the measurement flow path 98. In at least one example embodiment, the second sensor unit 102 may be installed between the first sensor unit 100 and the sample waste liquid accommodation unit 90 within the measurement flow path 98. The second sensor unit 102 may include a biosensor. In at least one example embodiment, the second sensor unit 102 may include an integrally molded enzyme sensor.

The second sensor unit 102 may include, for example, a glucose sensor 110 and/or a lactic acid sensor 112. Each of the glucose sensor 110 and the lactic acid sensor 112 may be placed in contact with the liquid flowing through the measurement flow path 98. The glucose sensor 110 may be configured to measure a glucose concentration of the liquid flowing through the measurement flow path 98. The lactic acid sensor 112 may be configured to measure a lactic acid concentration of the liquid flowing through the measurement flow path 98.

The second sensor unit 102 is not limited to an enzyme sensor. The second sensor unit 13 and may additionally, or alternatively, include a non-enzyme sensor.

The cleaning solution accommodation unit 88 and/or the sample waste liquid accommodation unit 90 may include medical bags, for example, in the same manner as the collection container 24 and the waste liquid accommodation unit 26 described above. In at least one example embodiment, the cleaning solution accommodation unit 88 and/or the sample waste liquid accommodation unit 90 may include tanks or the like, such as, prepared, for example, by a hard resin. The cleaning solution may be accommodated by the cleaning solution accommodation unit 88. The cleaning solution may be a liquid for the purpose of cleaning the second sensor unit 102. The cleaning solution may include, for example, a solution similar to the cleaning solution supplied from the aforementioned supply unit 22 to the culturing circuit 32.

The cell culturing device 12 may be configured to be set on the support device 14. The support device 14 may be configured to support the cell culturing device 12. The support device 14 may be a reusable product that is capable of being used a plurality of times.

As illustrated in FIGS. 1 and 2, the support device 14 may include a plurality of pumps 114 and a plurality of clamps 116. Each of the plurality of pumps 114 may impart a flowing force to the liquids inside the flow paths by squeezing the flow path wall parts of the cell culturing device 12. Although not specifically illustrated, it should be appreciated that, in at least one example embodiment, each of the plurality of pumps 114 may include a pressing member. The pressing member may include, for example, a rotating member and a plurality of pressing rollers. The plurality of pressing rollers may be attached to an outer circumferential portion of the rotating member. The plurality of pressing rollers may be arranged at intervals with spaces left therebetween in the circumferential direction of the rotating member. Each of the pressing rollers may rub against the outer surfaces of the flow path wall parts of the cell culturing device 12.

As illustrated in FIG. 1, the support device 14 may include two processing support units 142, two reactor support units 144 (see FIG. 2), one sampling support unit 146, a first sensor support unit 148, and a second sensor support unit 150.

In the following description, one of the two processing support units 142 may be referred to as a first processing support unit 152 and the other of the two processing support units 142 may be referred to as a second processing support unit 154. Further, with reference to FIG. 2, one of the two reactor support units 144 may be referred to as a first reactor support unit 156 and the other of the two reactor support units 144 may be referred to as a second reactor support unit 158.

As illustrated in FIGS. 1 and 2, the culturing circuit 32 of the first culture processing unit 80 may be capable of being attached to and detached from the first processing support unit 152. The culturing circuit 32 of the second culture processing unit 82 may be capable of being attached to and detached from the second processing support unit 154. The plurality of bioreactors 30 of the first culture processing unit 80 may be capable of being attached to and detached from the first reactor support unit 156. The plurality of bioreactors 30 of the second culture processing unit 82 may be capable of being attached to and detached from the second reactor support unit 158.

The first sensor unit 100 may be capable of being attached to and detached from the first sensor support unit 148. The second sensor unit 102 may be capable of being attached to and detached from the second sensor support unit 150.

The controller 16 may be configured to control the plurality of pumps 114, the plurality of clamps 116, the first sensor unit 100, and/or the second sensor unit 102.

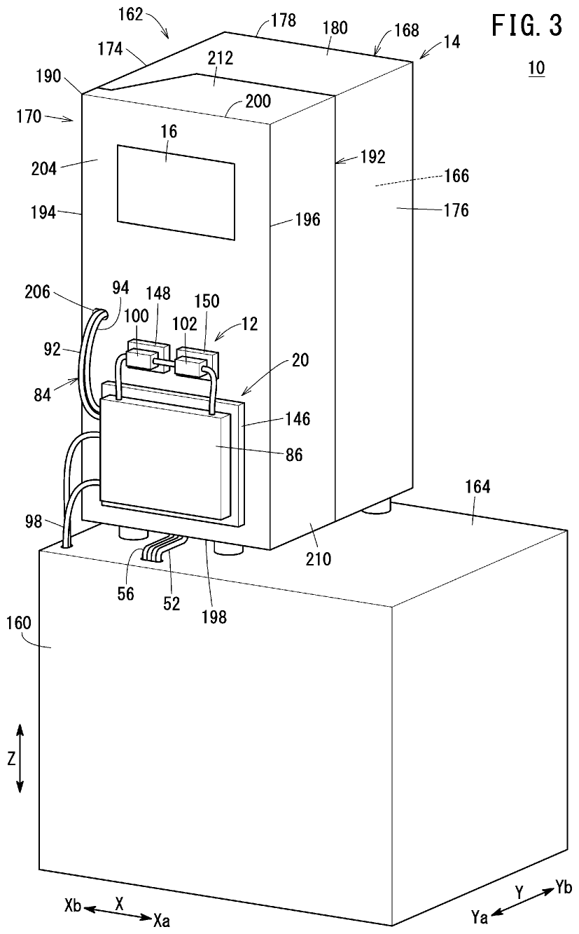

As illustrated in FIG. 3, the cell culturing system 10 may be arranged in a clean room. The support device 14 may be equipped with an accommodation case 160 and a housing 162.

The accommodation case 160 may be installed on a floor surface of the clean room. The accommodation case 160 may have a rectangular parallelepiped shape. Two of the supply units 22, two of the collection containers 24, two of the waste liquid accommodation units 26, the cleaning solution accommodation unit 88, and the sample waste liquid accommodation unit 90 may be accommodated inside the accommodation case 160.

The housing 162 may be arranged on a flat upper surface 164 of the accommodation case 160. A height dimension (i.e., the length in the direction of the arrow Z) of the housing 162 may be greater than a width dimension (i.e., the length in the direction of the arrow X) of the housing 162. The height dimension of the housing 162 may be greater than a thickness dimension (i.e., the length in the direction of the arrow Y) of the housing 162. In particular, the housing 162 may extend in a vertical direction.

As illustrated in FIGS. 3 and 4, the housing 162 may include an accommodation chamber 166 in which a portion of the cell culturing device 12 is accommodated. The culturing circuit 32 of each of the culture processing units 18 and the plurality of bioreactors 30 of each of the culture processing units 18 may be accommodated in the accommodation chamber 166. The housing 162 may include a non-illustrated temperature control device. Such a temperature control device may be configured to maintain the accommodation chamber 166 at an appropriate temperature. The housing 162 may include a housing main body 168 and a door portion 170.

As illustrated in FIG. 4, the housing main body 168 may include a bottom wall portion 172, a first side wall portion 174, a second side wall portion 176, a third side wall portion 178, and an upper wall portion 180. Each of the bottom wall portion 172, the first side wall portion 174, the second side wall portion 176, the third side wall portion 178, and the upper wall portion 180 may be defined by a flat plate portion. The first side wall portion 174 may extend upward in the direction of the arrow Xb from an end part of the bottom wall portion 172. The second side wall portion 176 may extend upward in the direction of the arrow Xa from an end part of the bottom wall portion 172. In at least one example embodiment, the first side wall portion 174 and the second side wall portion 176 may face toward each other. The third side wall portion 178 may extend upward in the direction of the arrow Yb from an end part of the bottom wall portion 172. The upper wall portion 180 may be connected to an upper end part of the first side wall portion 174, an upper end part of the second side wall portion 176, and an upper end part of the third side wall portion 178.

The housing main body 168 may include notched parts 182 therein. The notched parts 182 may be positioned at an end part in the direction of the arrow Ya of the housing main body 168. In at least one example embodiment, the notched parts 182 may include a first notched part 184, a second notched part 186, and a third notched part 188. The first notched part 184 may be positioned in the bottom wall portion 172 at a corner portion in the direction of the arrow Xa and the direction of the arrow Ya. The second notched part 186 may be positioned at the end of the second side wall portion 176 in the direction of the arrow Ya. The second notched part 186 may extend over the entire length in the vertical direction of the second side wall portion 176. The third notched part 188 may be positioned at the end of the upper wall portion 180 in the direction of the arrow Ya. A lower end of the second notched part 186 may communicate with the first notched part 184. An upper end of the second notched part 186 may communicate with the third notched part 188.

As illustrated in FIGS. 3 and 4, the door portion 170 may be installed on the housing main body 168 so that the accommodation chamber 166 is capable of being opened and closed. The door portion 170 may include a door portion main body 190 and a protective wall portion 192. The door portion main body 190 may be defined by a flat plate portion extending in a rectangular shape in the vertical direction. The door portion main body 190 may include a first long side portion 194, a second long side portion 196, a first short side portion 198, and a second short side portion 200.

The first long side portion 194 and the second long side portion 196 may extend in a vertical direction. The first long side portion 194 may be rotatably installed via a non-illustrated hinge or the like on the first side wall portion 174. The first short side portion 198 and the second short side portion 200 may extend in a horizontal direction. The first short side portion 198 may be positioned at a lower end part of the door portion main body 190. The second short side portion 200 may be positioned at an upper end part of the door portion main body 190.

The door portion main body 190 may have an inner surface 202 and an outer surface 204. In a state in which the door portion 170 is closed, the inner surface 202 of the door portion main body 190 may face toward the third side wall portion 178. The outer surface 204 of the door portion main body 190 may face in a direction opposite to the inner surface 202 of the door portion main body 190. The door portion main body 190 may include a through hole 206. The through hole 206 may extend from the inner surface 202 to the outer surface 204 of the door portion main body 190. The through hole 206 may be positioned in a central portion in the vertical direction of the door portion main body 190. The through hole 206 may be adjacent to the first long side portion 194.

The protective wall portion 192 may include a first protruding portion 208, a second protruding portion 210, and a third protruding portion 212. The first protruding portion 208 may protrude from the first short side portion 198 in the direction from the outer surface 204 toward the inner surface 202 of the door portion main body 190. The second protruding portion 210 may protrude in the direction in which the first protruding portion 208 protrudes from the second long side portion 196.

The third protruding portion 212 may protrude in the direction in which the first protruding portion 208 protrudes from the second short side portion 200. A lower end part of the second protruding portion 210 may be connected to the first protruding portion 208. An upper end part of the second protruding portion 210 may be connected to the third protruding portion 212. The third protruding portion 212 may extend over substantially the entire length of the second short side portion 200.

In a state in which the door portion 170 is closed, the protective wall portion 192 may be positioned in the notched parts 182 of the housing main body 168. In at least one example embodiment, in a state in which the door portion 170 is closed, the first protruding portion 208 may be positioned in the first notched part 184. In a state in which the door portion 170 is closed, the second protruding portion 210 may be positioned in the second notched part 186. In a state in which the door portion 170 is closed, the third protruding portion 212 may be positioned in the third notched part 188.

The first processing support unit 152, the second processing support unit 154, the first reactor support unit 156, the second reactor support unit 158, the sampling support unit 146, the first sensor support unit 148, and the second sensor support unit 150 may be installed in the housing 162.

As illustrated in FIG. 4, the first processing support unit 152 and the second processing support unit 154 may be fixed to the inner surface of the third side wall portion 178. The second processing support unit 154 may be positioned above the first processing support unit 152. The culturing circuit 32 of the first culture processing unit 80 may be mounted on the first processing support unit 152. The culturing circuit 32 of the second culture processing unit 82 may be mounted on the second processing support unit 154.

The first reactor support unit 156 and the second reactor support unit 158 may be fixed to the inner surface 202 of the door portion main body 190. The first reactor support unit 156 may be positioned in the vertical direction between the center of the door portion main body 190 and the first short side portion 198. The second reactor support unit 158 may be positioned in the vertical direction between the center of the door portion main body 190 and the second short side portion 200. The second reactor support unit 158 may be positioned above the first reactor support unit 156. In a state in which the door portion 170 is closed, the first reactor support unit 156 may face toward the first processing support unit 152. In a state in which the door portion 170 is closed, the second reactor support unit 158 may face toward the second processing support unit 154.

The plurality of bioreactors 30 of the first culture processing unit 80 may be mounted on the first reactor support unit 156. In a state in which the door portion 170 is closed, the plurality of bioreactors 30, which are mounted on the first reactor support unit 156, may be adjacent to the culturing circuit 32 of the first culture processing unit 80 that is mounted on the first processing support unit 152. The plurality of bioreactors 30 of the second culture processing unit 82 may be mounted on the second reactor support unit 158. In a state in which the door portion 170 is closed, the plurality of bioreactors 30, which are mounted on the second reactor support unit 158, may be adjacent to the culturing circuit 32 of the second culture processing unit 82 that is mounted on the second processing support unit 154.

The plurality of bioreactors 30 that are mounted on the first reactor support unit 156 and the second reactor support unit 158 may be covered by the protective wall portion 192. In at least one example embodiment, the second protruding portion 210 may cover from a horizontal direction the plurality of bioreactors 30 that are mounted on the first reactor support unit 156 and the second reactor support unit 158. The third protruding portion 212 may cover from above the plurality of bioreactors 30 that are mounted on the second reactor support unit 158. In accordance therewith, for example, when the door portion 170 is opened, it is possible to prevent the user from coming into contact with the plurality of bioreactors 30.

The sampling flow path 84 (e.g., the first introduction flow path 92 and the second introduction flow path 94) may be passed through the through hole 206 of the door portion main body 190. In greater detail, the sampling flow path 84 may be pulled out through the through hole 206 from the accommodation chamber 166 of the housing 162 to the exterior of the housing 162.

As illustrated in FIG. 3, the sampling support unit 146, the first sensor support unit 148, and the second sensor support unit 150 may be fixed to the outer surface 204 of the door portion main body 190. The sampling support unit 146 may be positioned in the vertical direction between the center of the door portion main body 190 and the first short side portion 198. The first sensor support unit 148 and the second sensor support unit 150 may be positioned above the sampling support unit 146. The first sensor support unit 148 and the second sensor support unit 150 may be adjacent to each other in the horizontal direction.

The sampling flow path 84 and the sampling circuit unit 86 may be mounted on the sampling support unit 146. The first sensor unit 100 may be mounted on the first sensor support unit 148. The second sensor unit 102 may be mounted on the second sensor support unit 150.

The controller 16 may be installed on an upper part of the outer surface 204 of the door portion main body 190.

In at least one example embodiment, the sampling support unit 146 is installed on the outer surface of the housing 162. As a result, the support device 14 in accordance with the present disclosure may be made more compact as compared, for example, to a case where a sampling support unit is arranged at a location separated away from a housing. Further, in accordance with at least one example embodiment, because the sampling flow path 84 may be short, the amount of the culture medium collected in the sampling unit 20 can be reduced as compared, for example, to a case where a sampling support unit is arranged at a location separation away from a housing.

The housing 162 may include the housing main body 168 and the door portion 170. The housing main body 168 may include the accommodation chamber 166. The door portion 170 may be installed on the housing main body 168 and may be configured to open and close the accommodation chamber 166. The sampling support unit 146 may be installed on the outer surface 204 of the door portion 170.

In accordance with such a configuration, mounting of the sampling circuit unit 86 on the sampling support unit 146 can be easily performed. Further, it is possible for the user to easily access the sampling unit 20.

The support device 14 may be equipped with the reactor support units 144 to which the plurality of bioreactors 30 are capable of being attached and detached. The reactor support units 144 may be fixed to the inner surface 202 of the door portion 170.

In accordance with such a configuration, while the sampling flow path 84 can be made short, the culture medium, which is guided out from the plurality of bioreactors 30, can be collected in the sampling unit 20.

The door portion 170 may include the through hole 206. The sampling flow path 84 may be passed through the through hole 206.

In accordance with such a configuration, the sampling flow path 84 can be made even shorter.

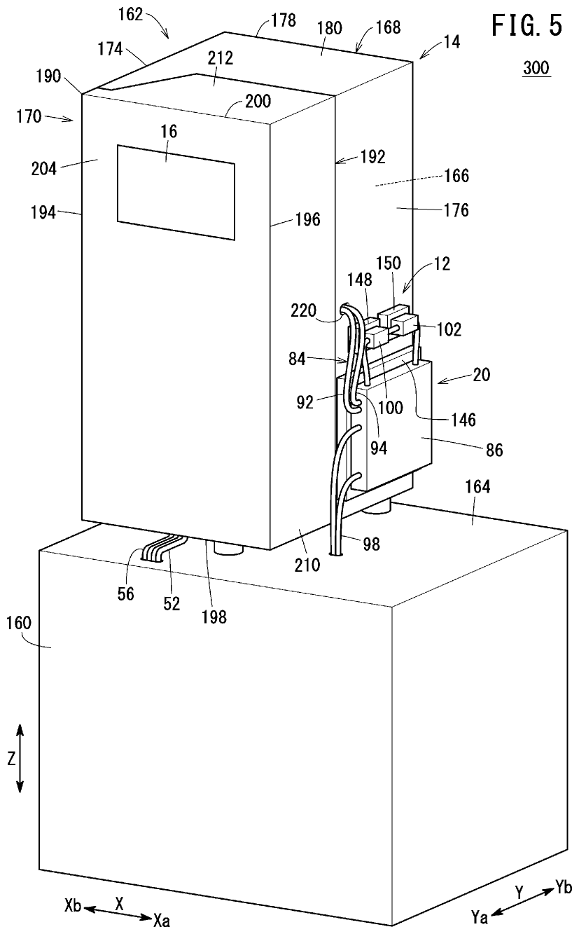

FIG. 5 illustrates an example cell culturing system 300. In the following discussion concerning the cell culturing system 300, constituent elements that may be the same as those of the cell culturing system 10 described above in the context of FIGS. 1-4 are designated by the same reference numerals and description of such features is omitted.

As illustrated in FIG. 5, in the cell culturing system 300, the sampling support unit 146, the first sensor support unit 148, and the second sensor support unit 150 may be fixed to an outer surface (e.g., a surface facing in the direction of the arrow Xa) of the second side wall portion 176 of the housing main body 168. The sampling support unit 146 may be positioned between the center of the outer surface of the second side wall portion 176 in the vertical direction and a lower end of the second side wall portion 176.

The first sensor support unit 148 and the second sensor support unit 150 may be positioned above the sampling support unit 146. The first sensor support unit 148 and the second sensor support unit 150 may be adjacent to each other in the horizontal direction. The second side wall portion 176 may include a through hole 220. The through hole 220 may be positioned in a central portion in the vertical direction of the second side wall portion 176. The through hole 220 may penetrate in a thickness direction through the second side wall portion 176.

In at least one example embodiment, the sampling support unit 146 may be installed on the outer surface of the housing main body 168. Therefore, the support device 14 in accordance with the present disclosure may be made more compact as compared, for example, to a case in which a sampling support unit is arranged at a location separated away from a housing. Further, in accordance with at least one example embodiment, because r the sampling flow path 84 may be short, the amount of the culture medium collected in the sampling unit 20 may be reduced as compared, for example, to a case in which a sampling support unit is arranged at a location separated away from a housing.

Within the outer surface of the housing main body 168, the sampling support unit 146 may be installed on a side surface (e.g., the outer surface of the second side wall portion 176) which together with being adjacent to the door portion 170, may be oriented in the horizontal direction.

In accordance with such a configuration, mounting of the sampling circuit unit 86 on the sampling support unit 146 can be easily performed. Further, it is possible for the user to easily access the sampling circuit unit 86.

The through hole 220 in order for the sampling flow path 84 (e.g., the first introduction flow path 92 and the second introduction flow path 94) to be passed therethrough may open on the outer surface of the second side wall portion 176.

In accordance with such a configuration, the sampling flow path 84 can be made even shorter.

The present invention is not limited to the embodiments described above, and various alternative configurations could be adopted therein without deviating from the essence and gist of the present invention.

The sampling support unit 146, the first sensor support unit 148, and the second sensor support unit 150 may be installed on the outer surface of the first side wall portion 174 of the housing main body 168, on the outer surface of the upper wall portion 180 of the housing main body 168, or on the outer surface of the third side wall portion 178. Within the outer surface of the housing 162, the first sensor support unit 148 and the second sensor support unit 150 may be installed on a surface that differs from the surface on which the sampling support unit 146 is installed. The sampling support unit 146, the first sensor support unit 148, and the second sensor support unit 150 may be integrally molded products.

The sampling unit 20 may be connected to any one of the first waste liquid flow path 70, the second waste liquid flow path 72, or the third waste liquid flow path 74. Each of the culture processing units 18 may include only one of the bioreactors 30. The cell culturing device 12 may include only one of the culture processing units 18. The cell culturing device 12 may also include three or more of the culture processing units 18.

In at least one example embodiment, the support device 14 may support the cell culturing device 12 that includes the culturing unit 28. The culturing unit may include the bioreactors 30 in which the cells are cultured and the sampling unit 20 that collects from the culturing unit the culture medium used for culturing the cells. The sampling unit 20 may include the sampling circuit unit 86 and the sampling flow path 84 that mutually connects the culturing unit and the sampling circuit unit. The support device may include the housing 162 that includes the accommodation chamber 166 in which the culturing unit is accommodated and the sampling support unit 146 to which the sampling circuit unit is capable of being attached and detached. The sampling support unit 146 may be installed on the outer surface 204 of the housing 162.

In at least one example embodiment, the housing 162 may include the housing main body 168 in which the accommodation chamber 166 may be included and the door portion 170 installed on the housing main body 168, and which opens and closes the accommodation chamber 166. The sampling support unit 146 may be installed on the outer surface 204 of the door portion 170.

In at least one example embodiment, the support device 14 may include the reactor support unit 144 to which the bioreactors may be capable of being attached and detached. The reactor support unit 144 may be fixed to the inner surface 202 of the door portion 170.

In at least one example embodiment, the door portion 170 may include the through hole 206 in order for the sampling flow path 84 to be passed therethrough.

In at least one example embodiment, the housing 162 may include the housing main body 168 in which the accommodation chamber 166 is included. The door portion 170 may be installed on the housing main body 162. The door portion 170 may be configured to open and close the accommodation chamber 166. The sampling support unit 146 may be installed on the outer surface of the housing main body 168.

In at least one example embodiment, within the outer surface of the housing main body 168, the sampling support unit 146 may be installed on a side surface which is oriented in a horizontal direction together with being adjacent to the door portion 170.

In at least one example embodiment, the side surface may include the through hole 220 in order for the sampling flow path 84 to be passed therethrough.

1. A support device for a cell culturing device, the support device comprising:
a housing configured to receive a culturing unit of the cell culturing device; and
a sampling support unit couplable to the housing and configured to receive a sampling circuit unit of the cell culturing device.

2. The support device of claim 1, wherein the housing includes an accommodation chamber configured to receive the culturing unit.

3. The support device of claim 2, wherein the housing further includes:
a door portion configured to move from an open position to a closed position, wherein in the open position the accommodation chamber is accessible and in the closed position the accommodation chamber is inaccessible.

4. The support device of claim 3, wherein the door portion has an exterior-facing surface and an opposing interior-facing surface, the interior-facing surface facing the accommodation chamber in the closed position, the sampling support unit being couplable to the exterior-facing surface of the door portion.

5. The support device of claim 4, wherein the support device further includes:
a reactor support unit configured to receive a bioreactor of the culturing unit, the bioreactor configured to culture cells.

6. The support device of claim 5, wherein the reactor support unit is couplable to the interior-facing surface of the door portion.

7. The support device of claim 3, wherein the door portion includes a through hole, the through hole configured to receive a sampling flow path of the cell culturing device, the sampling flow path configured to connect the culturing unit and the sampling circuit unit.

8. The support device of claim 3, wherein the sampling support unit is couplable to a side surface oriented in a horizontal direction of the housing, the side surface being adjacent to the door portion.

9. The support device of claim 8, wherein the side surface includes a through hole, the through hole configured to receive a sampling flow path of the cell culturing device, the sampling flow path configured to connect the culturing unit and the sampling circuit unit.

10. A cell culturing system comprising:
a cell culturing device including:
a culturing unit configured to culture cells using a culture medium, and
a sampling unit configured to receive the culture medium from the culturing unit; and
a support device including:
a housing configured to receive the culturing unit; and
a sampling support unit couplable to the housing and configured to receive at least a portion of the sampling unit.

11. The cell culturing system of claim 10, wherein the sampling unit includes:
a sampling circuit unit, and
a sampling flow path configured to connect the culturing unit and the sampling circuit unit.

12. The cell culturing system of claim 11, wherein the sampling support unit is configured to receive the sampling circuit unit.

13. The cell culturing system of claim 10, wherein the housing includes:
an accommodation chamber configured to receive the culturing unit; and
a door portion configured to move from an open position to a closed position, wherein in the open position the accommodation chamber is accessible and in the closed position the accommodation chamber is inaccessible.

14. The support device of claim 13, wherein the door portion has an exterior-facing surface and an opposing interior-facing surface, the interior-facing surface facing the accommodation chamber in the closed position, the sampling support unit being couplable to the exterior-facing surface of the door portion.

15. The support device of claim 14, wherein the support device further includes:
a reactor support unit configured to receive a bioreactor of the culturing unit, the bioreactor configured to culture cells.

16. The support device of claim 15, wherein the reactor support unit is couplable to the interior-facing surface of the door portion.

17. The support device of claim 13, wherein the door portion includes a through hole, the through hole configured to receive a sampling flow path of the cell culturing device, the sampling flow path configured to connect the culturing unit and the sampling circuit unit.

18. The support device of claim 13, wherein the sampling support unit is couplable to a side surface oriented in a horizontal direction of the housing, the side surface being adjacent to the door portion.

19. The support device of claim 19, wherein the side surface includes a through hole, the through hole configured to receive a sampling flow path of the cell culturing device, the sampling flow path configured to connect the culturing unit and the sampling circuit unit.

20. A support device for a cell culturing device, the support device comprising:
a housing configured to receive a culturing unit of the cell culturing device, the housing including:
a door portion configured to move from an open position to a closed position; and
a sampling support unit couplable to the housing and configured to receive a sampling circuit unit of the cell culturing device, the sampling support unit couplable to the door portion of the housing or to a side surface oriented in a horizontal direction of the housing, the side surface being adjacent to the door portion.
